(19) United States
(12) Plant Patent Application Publication
(10) Pub. No.: US 2018/0160583 P1
(43) Pub. Date: Jun. 7, 2018

(54) Asian plum tree named 'Sweet Pekeetah'

(50) ;

(71) Applicant: Universidad De Chile, Santiago (CL)

(72) Inventor: Rodrigo Arturo Infante Espiñeira, Vitacura (CL)

(21) Appl. No.: 15/732,605

(22) Filed: Dec. 1, 2017

(30) Foreign Application Priority Data
Dec. 6, 2016 (CL) 1664

(51) Int. Cl. A01H 5/08

(52) U.S. Cl. PLT/184

(57) ABSTRACT

A new and distinct plum variety is described. ‘Sweet Pekeetah’ is an Asian plum variety with unique traits, as extremely late blooming and ripening dates. The flesh of ‘Sweet Pekeetah’ has crunchy texture, green color and soluble solids concentration reaches 20-22%, with upstanding sensory quality. The postharvest life potential of ‘Sweet Pekeetah’ can last 60 days keeping the original high sensory quality.

### BACKGROUND

Seedlings obtained from collecting seed from open pollinated fruit during controlled hybridization. This new cultivar resulted from a controlled hybridization between the Asian plum genotype known as ‘213-UR-056’ (female progenitor) and the Asian plum genotype ‘178-R-01’ (Male progenitor). Both progenitors are non-patented genotypes that are currently used as recurrent parental in the breeding program of the University of Chile. The sexual crossing was performed in 2008 at Rinconada de Maipú, Metropolitan Region, Chile (latitude −33° 30′S, longitude −70° 48′W, altitude 654 m above sea level). This genotype was for the first time asexually propagated through grafting on ‘Nemaguard’ rootstock in the same field on 2011. The rootstock ‘Nemaguard’ is a chance seedling selected in 1959 by USDA, and it has never been patented. The invention described herein relates to the discovery and the asexual propagation through grafting on ‘Nemaguard’ rootstock of a new variety of Asian plum tree, Prunus salicina named ‘Sweet Pekeetah’. The resulting plants propagated true to type, demonstrating that the characteristics of the new variety are stable and transmitted without change through succeeding generations.

### SUMMARY

Under the Chilean growing conditions ‘Sweet Pekeetah’ is distinguished from varieties of common knowledge such as ‘Angeleno’ and ‘Friar’ by the following characteristics:

The ripening period of ‘Angeleno’ is three to four weeks before that of the ‘Sweet Pekeetah’. Additionally, the fruit of ‘Sweet Pekeetah’ has higher soluble solids concentration and lower softening rate than the variety ‘Angeleno’. Compared with ‘Angeleno’, ‘Sweet Pekeetah’ shows a firmer and more crunchy flesh, the fruit size is 140-150 gr. ‘Angeleno’ plum is harvested in Chile on approximately February 20^{th} and ‘Sweet Pekeetah’ is harvested 15 to 20 days after.

The fruit of ‘Friar’ are moderately asymmetric and the shape of apex is depressed, conversely the fruit of ‘Sweet Pekeetah’ are symmetric and the shape of apex is rounded. The color of flesh of ‘Friar’ is yellow and the color of flesh of ‘Sweet Pekeetah’ is yellowish green. ‘Friar’ plum is harvested in Chile on approximately January 20^{th}, and ‘Sweet Pekeetah’ is harvested 35 to 40 days after.

### BRIEF DESCRIPTION OF THE DRAWINGS

FIG. 1 is a photograph showing a typical specimen of the foliage and fruit of the new variety depicted in colors as nearly true as is reasonably possible to make the same in a color illustration of this character. The observations were made on mature trees which ranged in age between four and five years old, four years after budding. The photograph shows the ‘Sweet Pekeetah’ fruit just after harvest.

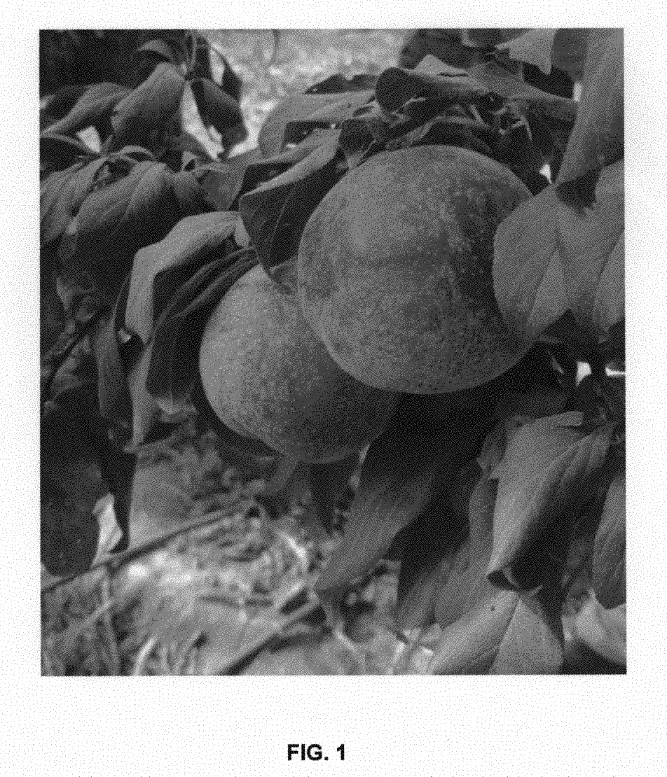

FIG. 2 shows a tree of ‘Sweet Pekeetah’, with height, foliage and fruit of the new variety depicted in colors as nearly true as is reasonably possible to make the same in a color illustration of this character.

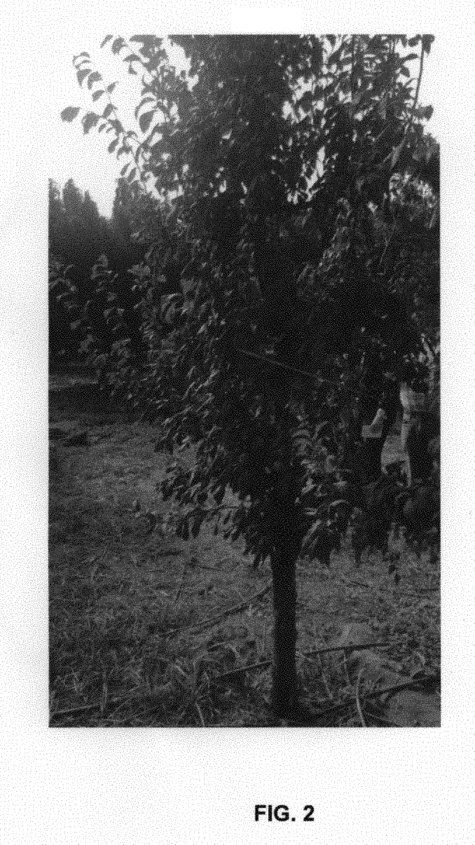

FIG. 3 shows a branch of ‘Sweet Pekeetah’ tree showing fruits and leaves as nearly true as is reasonably possible to make the same in a color illustration of this character.

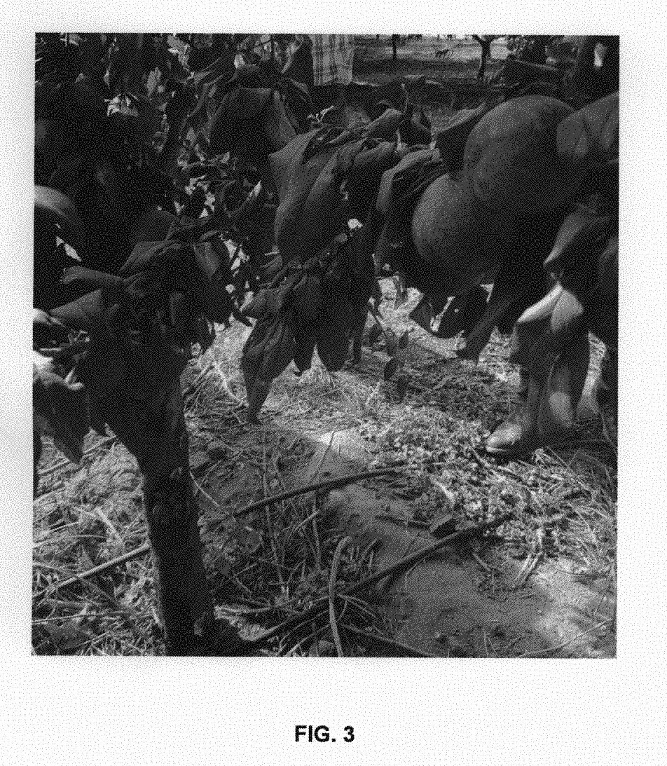

### DETAILED DESCRIPTION

The ‘Sweet Pekeetah’ is a self-unfertile, with semi erect plant growth, weak to medium vigor. The fruit is round, symmetrical, with a deep purple skin color that covers 80 to 90% of the surface over a green background color.

Blooming time of ‘Sweet Pekeetah’ is extremely late, being contemporary to some European plums as ‘French prune’, which is three to four weeks after the majority of known Asian plum varieties.

The main feature of ‘Sweet Pekeetah’ is its slow pulp softening rate commencing during the last two weeks before the commercial harvest. This characteristic enables delaying the harvest, to increase the size of the fruit and the soluble solids content without affecting their post-harvest life potential.

Secondly, ‘Sweet Pekeetah’ shows a high post-harvest life, which can reach up to 60 days at 0° C. while maintaining its initial sensory quality. From a sensory point of view, this is a variety of medium acidity with a balanced sweetness/acidity ratio and in general an upstanding sensory quality. The variety is not susceptible to “chilling injury” and therefore can be suitable for exporting to distant markets from the production zone. Another distinguishing factor compared to other known varieties is that the ‘Sweet Pekeetah’ reaches a high soluble solid concentration, which can scores 20 to 22%, while ‘Angeleno’, for example, barely reaches 16%.

The flesh of ‘Sweet Pekeetah’ when picked shows a brilliant green color, which is also a remarkable and not common characteristic among this species. The green color of the flesh remains for the first 20 days when fruit is maintained in cold chamber, but after that period, it began to change to yellow-orange. In term of texture, ‘Sweet Pekeetah’ has an uncommon crunchiness at harvest and even after 60 days of cold storage.

The following is a brief description of the new variety with color terminology in accordance with CIELab coordinates.

The observations were made on mature trees which ranged in age between four and five years old. Trees were grown under standard orchard practices.
Tree:
   Type of bearing.—On spurs and long shoots, similar to cultivar ‘Angeleno’ and ‘Shiro’.
   Vigor.—Weak, similar to cultivar ‘Black Gold’ and ‘Satsuma’, shoots reach 40 to 60 cm and shoot thickness reach 4.5-5.0 mm diameter on a regular spring/summer growing period.
   Habit.—Semi-upright, similar to cultivar ‘Laroda’. The size of the tree is medium, reaching 3.5-3.9 m high shaped as an open vase, 2.0-2.2 m diameter at the fifth year.
   One-year-old shoot, color.—Brown, similar to cultivar ‘Methley’, the objective color in CIELab coordinates is L=26.40 a=3.37 b=7.51 (Mcguire R. G., 1992).
   Spur length.—Medium, similar to cultivar ‘Frontier’ and the flowering shoot thickness reach 3.0-5.3 cm diameter and 8-10 cm length.
   Vegetative bud.—Size: Small, similar to cultivar ‘Harry Pickstone’, reaching 5-7 mm length.
   Vegetative bud.—Shape of apex: Acute, similar to cultivar ‘Eldorado’.
   One-year-old shoot position of vegetative bud in relation to shoot.—Adpressed, similar to cultivar ‘Queen Ann’.
   Leaf blade length.—Medium, reaching 12.8 cm length, similar to cultivar ‘Taiyou’.
   Leaf blade width.—Medium, reaching 5.5 cm width, similar to cultivar ‘Sordum’.
   Leaf blade, length/width ratio.—Moderately elongated, reaching in average 2.3, similar to cultivar ‘Pioneer’.
   Leaf blade shape.—Elliptic, similar to cultivar ‘Black Gold’ and ‘Taiyou’.
   Leaf blade color of upper side.—Medium green, like cultivar ‘Abundance’, classified in CIELab coordinates: L=37.45 a=−8.20 b=−15.48 (Mcguire R. G., 1992).
   Leaf blade angle of apex (excluding tip).—Acute, like cultivar ‘Taiyou’.
   Leaf glossiness of upper side.—Medium, similar to cultivar ‘Frontier’.
   Leaf blade density of pubescence of lower side.—Sparse, similar to cultivar ‘Angeleno’.
   Leaf blade incisions of margin.—Serrate, like cultivar ‘Dapple Dandy’.
   Petiole length.—Medium, similar to cultivar ‘Frontier’, reaching 1.3 cm length.
   Leaf position of nectaries.—Predominantly on base of leaf blade, like cultivar ‘Methley’.
   Pedicel length.—Medium, reaching approximately 10 mm length, similar to cultivar ‘Queen Ann’.
Flower:
   Diameter.—Medium, reaching 15-17 mm, like cultivar ‘Shiro’.
   Arrangement of petals.—Free, similar to cultivar ‘Laroda’.
   Sepal shape.—Narrow elliptic, similar to cultivar ‘Laroda’.
   Petal length.—Medium, like cultivar ‘Santa Rosa’.
   Petal shape.—Obovate, similar to cultivar ‘Mammoth Cardinal’.
   Petal undulation of margin.—Medium, like cultivar ‘Queen Ann’.
   Stigma position in relation to anthers.—Below, similar to cultivar ‘Mariposa’.
Fruit:
   Length of stalk.—Medium, reaching in average 8.9 mm, similar to cultivar ‘Sordum’.
   Size.—Medium, like cultivar ‘Shiro’, reaching 140-150 gr.
   Height.—Medium, reaching in average 55.4 mm similar to cultivar ‘Harry Picktone’.
   Width.—Medium, diameter in line with suture plane, approximately 60 mm. Diameter perpendicular to suture plane 65 mm, similar to cultivar ‘Casselman’.
   Shape in lateral view.—Oblate, similar to cultivar ‘Friar’.
   Symmetry.—Slightly asymmetric, like cultivar ‘Laroda’.
   Shape of base.—Depressed, similar to cultivar ‘Calita’.
   Shape of apex.—Rounded, similar to cultivar ‘Shiro’.
   Depth of stalk cavity.—Medium, reaching in average 9.0 mm, like cultivar ‘Angeleno’.
   Width of stalk cavity.—Medium, reaching approximately 12 mm similar to cultivar ‘Beni Ryozhen’.
   Depth of suture.—Medium, similar to cultivar ‘Sordum’.
   Bloom of skin.—Strong, like cultivar ‘Sordum’.
   Ground color of skin.—Yellowish green, like cultivar ‘Taiyou’.
   Relative area of over color.—Very large, like cultivar ‘Friar’.
   Over color of skin.—Purple, classified in CIELab coordinates: L=535.14 a=127.76 b=53.44 (Mcguire R. G., 1992), like cultivar ‘Karari’.
   Pattern of over color.—Solid flush only, similar to cultivar ‘Friar’.
   Number of lenticels.—Medium, like cultivar ‘Sunrise’.
   Size of lenticels.—Medium, like cultivar ‘Extreme’.
   Color of flesh.—Yellowish green, the objective color in CIELab coordinates is L=631.83 a=−93.47 b=353.28 (Mcguire R. G., 1992), similar to cultivar ‘Shiro’.
   Firmness.—Firm, reaching in average 7-8 pounds, like cultivar ‘Laroda’ and ‘Taiyou’.
   Juiciness.—High, like cultivar ‘Reubennel’.
   Acidity.—Low, reaching in average 1.1% malic acid when ripe, similar to cultivar ‘Angeleno’.
   Sweetness.—High, reaching in average 20-22° Brix when ripe, like cultivar ‘Black Gold’.
   Adherence of stone to flesh.—Non-adherent, similar to cultivar ‘Fortune’.
   Amount of fiber.—Low.
Stone:
   Size.—Small, 2.0 cm long and 1.6 cm width, like cultivar ‘Angeleno’.
   Shape in lateral view.—Medium elliptic, like cultivar ‘Santa Rosa’.
   Shape in ventral view.—Medium elliptic, like cultivar ‘Santa Rosa’.
   Shape in basal view.—Medium elliptic, like cultivar ‘Bragialla’.
   Symmetry in lateral view.—Symmetric, similar to cultivar ‘Angeleno’.
   Texture of lateral surfaces.—Granular, similar to cultivar ‘Nubiana’.
   Width of stalk-end.—Medium, like cultivar ‘Harry Pickstone’.
Phenology:
   Time of beginning of flowering.—Very late, 2 weeks later than ‘Angeleno’. In the Southern Hemisphere occurs between 10^{th} to 20^{th} September.
   Time of beginning of fruit ripening.—Very late, 3 weeks later than ‘Angeleno’. In the Southern Hemisphere occurs between 10^{th} to 20^{th} March.
Keeping quality: After 60 days in cold storage + 3 days at room temperature keeps a glossy flesh, good taste and crunchy flesh.

What is claimed is:

1. A new and distinct plum tree substantially as illustrated and described.
